(19) United States
(12) Patent Application Publication
(10) Pub. No.: US 2004/0202684 A1
(43) Pub. Date: Oct. 14, 2004

(54) Personalized interactive natural cosmetics

(76) Inventor: David Djerassi, New York, NY (US)

Correspondence Address: GIBBONS, DEL DEO, DOLAN, GRIFFINGER & VECCHIONE, 1 RIVERFRONT PLAZA, NEWARK, NJ, 07102-5497, US

(21) Appl. No.: 10/409,578

(22) Filed: Apr. 8, 2003

(51) Int. Cl. A61K 7/00

(52) U.S. Cl. 424/401

(57) ABSTRACT

A kit and method for consumer preparation of customized and personalized topical cosmetic products which contain a mixing vessel and separate component systems, each of said component systems being different and when blended in the mixing vessel form a cosmetic which, if desired, can be blended with natural food or beverage materials and can be applied from the mixing vessel with or without the natural material blend to the human body.

### FIELD OF INVENTION

The field of the invention relates to cosmetic compositions which can be personally formulated right before topical application.

### BACKGROUND OF THE INVENTION

Over the last fifteen years, many manufacturers and marketers have introduced anti-aging skin care products claiming rejuvenation of the skin. Many of these products are fortified with vitamins, antioxidants, natural extracts and in some cases exotic ingredients. The reasons for such additions have been the consumer's interest in health enhancing cosmetics and the emerging data on the benefits associated with the use of such ingredients in topical products. Such benefits have also spilled-over into other segments of cosmetics & toiletries including hair care, nail care, make-up, oral care, etc.

The many documented benefits of these cosmetic compositions containing vitamins, natural extract materials and therapeutically active topical skin products include protection against the environmental hazards and correction of the changes caused by both chronological aging and photoaging. However, such benefits are dependent on many factors, in particular the levels of cosmetic active materials, the correct blends of such active materials , their stability in the products, the delivery vehicle and the packaging used. In addition, many of these vitamins and natural extracts are unstable and deteriorate upon storage. Therefore, unfortunately, many of the products currently on the market do not meet the above requirements. Some contain extremely low levels of the important vitamins and Natural Extracts. Others suffer from inadequate stability, which significantly reduces their performance over time. In addition, the right blend of cosmetically active materials such as vitamins and natural extracts can vary with the person and the skin condition of the person. Since commercial cosmetic products are not formulated to individual needs, in general, they do not always contain the right blend and the right vehicles for enhanced product performance in a given person.

Hence there is a strong need for high potency and high performance cosmetics in general and for skin care products adapted for individual needs. In particular, such products should be formulated with active ingredients at proper levels with the right blends and in special delivery vehicles for optimal performance and stability and to meet individual needs. In this regard, there is a need for cosmetic products to be made just before their use and application, with natural ingredients such as freshly squeezed fruits and vegetables, freshly brewed teas, coffee, milk and red wine and blends thereof depending upon the needs of a given individual. Such fresh cosmetic products that are rich blends of vitamins, minerals, antioxidants, carotenoids provide enhanced performance as compared to the performance of many of today's commercially available cosmetic products which not always contain adequate amounts of these natural ingredients.

### SUMMARY OF THE INVENTION

In accordance with this invention, a kit and a method for preparing a personalized cosmetic compositions by the consumer has been developed. In accordance with this invention, the consumer is supplied with a kit that allows him to prepare the cosmetics in a customized matter just prior to application and if desired in preparing these cosmetics, the consumer can add various natural materials derived from foods and beverages. The cosmetic composition can be prepared by the consumer by mixing various component systems together with or without the natural materials in situ immediately prior to application to the skin to form a ready to use cosmetic. In this manner, the freshness of the cosmetic product is preserved and the performance is enhanced.

The kit which is sold to the consumer in accordance with this invention comprises two or three separated containers each containing a predetermined amount of a component system formed from different mixtures of ingredients. These component systems are present in the kit in a predetermined amount such that when all said ingredients in these component systems are mixed together, they form the desired cosmetic composition which can be directly topically applied to the human body. The kit contains a mixing vessel for mixing said component systems in the predetermined ratio to form this composition from which it can be directly topically applied. The first component system contains a mixture of cosmetically active ingredients. The second component system contains a liquid aqueous cosmetic carrier medium. The third component system contains a thickening agent which when added to the liquid medium forms the liquid topical product such as a lotion, shampoo, conditioner, rinse, or cream. In accordance with one embodiment of this invention, the kit contains two separate containers, one of which contains the first and third components while the other container contains the second component system. In this manner the first and third component systems are separated from the second component system by means of two containers. This allows one to separate the active ingredients from the liquid aqueous cosmetic carrier medium, which causes lost of activity and deterioration of these cosmetically active ingredients.

In accordance with another embodiment of this invention, the kit contains three separated containers. Each contains one of the specific component systems. In this manner, the three component systems are separated from each other so that there is no interaction of the cosmetically active ingredients in the first container with either the thickening agent in the third container or the liquid aqueous cosmetic carrier medium in the second container. Such a three container system provides for maximum stability and activity of the cosmetically active ingredients just prior to mixing. In addition it allows increased levels of active ingredients to be incorporated in the first container without fear of loss of activity by means such as decomposition. By proving the thickening agent in a separate compartment or container also allows one to thicken the composition to any desired level.

With respect to both embodiments, the three component systems, provided in either two or three containers, can be mixed by the consumer and to this mixture the consumer may add a fourth liquid component system. This fourth liquid component system contains liquid or liquid extracts derived from food materials (such as a fruit or vegetable) or beverage materials or mixtures thereof. This fourth component system can be added to the mixing vessel either before or after the mixing of the three component systems in the mixing vessel or while the three component systems are mixed. In this manner, the liquid food or beverage materials or mixtures thereof can be freshly prepared to the special needs of the consumer and added to the cosmetic composition without danger of decomposition of these natural materials which generally occurs over time.

### DETAILED DESCRIPTION

In accordance with this invention, a kit is provided for allowing the consumer to customize and personalize cosmetic compositions. This kit includes three component systems, packaged in two or three separate containers, and a mixing vessel which allows the three component systems to be mixed to form the cosmetic composition prior to topical application to the human body. While the kit can be used for application to the human body, it can also be used for application to the body of pets such as dogs, cats, horses, etc.

The use of the kit of this invention provides a new method for forming and applying a liquid cosmetic composition topically to the body of a human. The liquid product can be applied topically to any part of the human body such as the skin, hair or nails in topical cosmetic liquid forms such as a lotion, shampoo, conditioner, rinse, or cream. This method involves providing three component systems which when mixed together form the topical cosmetic product such as a cream or lotion for application to the human body. These component systems, are packaged in at least two separated containers. The first component system is a mixture of cosmetically active ingredients. The second component system, which is kept separate from the first and third component systems, is a liquid aqueous cosmetic carrier medium. The third component system which can be combined and packaged with the first component system comprises a thickening or gelling agent or mixture of thickening and gelling agents which when added to the liquid medium form the desired liquid cosmetic product. In this manner, the three component systems contained in each of these packages are mixed in the mixing vessel supplied by the kit in a predetermined ratio to form the desired liquid cosmetic product. After mixing, the liquid cosmetic product containing the cosmetic ingredients is ready for topical application to body such as the skin, nails or hair directly from the mixing vessel.

In accordance with the preferred method and kit for use in this invention, the three component system is provided in three separate containers. In this manner, there is no chance of lost of activity of the cosmetically active ingredients prior to mixing through the presence of the thickening agent or the liquid aqueous cosmetic carrier base. While the three components can be placed in two separate containers with first and third components mixed, this is not preferred. In accordance with this invention, it is important to keep the second component which constitutes the liquid aqueous cosmetic carrier medium separate from the first component containing the cosmetically active ingredients. However, it is generally preferred for best results to provide these three component systems as three separate component systems prior to mixing. By providing the thickening agent in a separate compartment or container also allows one to thicken the composition to any desired level.

In accordance with a preferred embodiment of this invention, during the mixing step or before or after the mixing of the component systems, a fourth component system containing liquid forms or extracts of natural materials used for food and beverages can be mixed with the other three component systems in forming the cosmetic. In such manner, a cosmetic composition is provided which can be formulated at home and can contain fresh, naturally based balanced and effective ingredients. The cosmetic products which are produced in this manner can be tailored to individual needs of the user according to their skin type, skin problem, age, time or day or night, season, etc. It is important that the second component system, as outlined above, be kept in a separated compartment or individual capsule or package from the first and third component systems. These three component systems can be blended alone or together with one or more natural materials to form the elegant, finished cosmetic product just prior to use. It has been found that many of the cosmetically active materials and the natural materials are unstable in storage especially in the presence of water. Mixing with water and/or storage for long periods of time can cause deactivation of these natural materials and/or cosmetically active materials due to decomposition or deterioration over time.

As can be seen from this invention, the preparation of the cosmetic products is simple since only mixing need be utilized. Since the products can be mixed at ambient room temperature unlike most of the commercially available cosmetic products such as creams and lotions which require high temperatures, deactivation of the ingredients is prevented. Therefore, this invention provides a means whereby deactivation of the active ingredients and natural materials such as through deterioration or decomposition before use can be prevented so that a fresh cream, lotion or other liquid cosmetic product can be produced for topical application without loss of the stability and potency of the active ingredients and natural materials.

In accordance with this invention the first component contains any conventional cosmetically active material which provides a therapeutic or beneficial cosmetic action to the human body particularly the skin, hair or nails. In accordance with this invention the cosmetically active materials that may be incorporated into the first component system include anti-aging ingredients, anti-wrinkling agents, minerals, preservatives, antioxidants, moisturizers, vitamins, sunscreen agents, and anti-acne agents. Among the preferred cosmetically active material are vitamins such as Vitamin A, B₁, B₂, B₃, B₅, B₆, B₁₂, biotin, Vitamin C, Vitamin D, Vitamin E, Folic Acid and Vitamin K, as well as flavonoids, and anthocynines. Other preferred cosmetically active ingredients which can be utilized in the first component system include selective pure herbal extracts, antioxidants such as flavonoids and phytols, minerals such as calcium, copper, chromium, magnesium, manganese, phosphorous, potassium, selenium or sodium, anti-wrinkling agents, anti-acne agents, lutein, lycopene, moisturizers such as Vitamin E, mineral oils, diisopropyl adipate, cetearyl octanoate. Preferred antioxidants are phytols and flavonoids. The first component system can include as a cosmetic active ingredient sunscreen agents commonly employed in conventional preformed cosmetic compositions.

Many of these active materials, particularly vitamins, minerals, and antioxidants are relatively unstable and are subject to loss of activity including by decomposition when incorporated into finished conventional cosmetic preparations. Therefore, in accordance with this invention, the first component system containing the cosmetically active materials should be substantially free from water which can cause loss of activity including decomposition of these active ingredients. Therefore, the first component system of this three component system should be generally substantially free from water. It may contain oils which either provide natural body moisturizing and/or act as the liquid medium for the active ingredients such as Vitamin A and Vitamin E, Vitamin C, etc. In general, the first component system is provided in an amount of from about 0.5% to about 20% of the final cosmetic composition which is prepared there from. The cosmetically active ingredients in the first component system may be varied depending upon whether the cosmetic is used for a day or night treatment, or for conditioning or cleansing of body such as the skin hair or nails. For a day treatment, generally moisturizers and/or antioxidants are preferred as the cosmetically active materials. For night treatment, anti-wrinkling agents and anti-aging agents are preferred in these compositions.

The first component system containing the cosmetically active ingredients set forth above should be substantially free of water. That means, only small amounts of water of less than 5.0% by weight percent, more preferably, less than about 2.0% weight, based upon the weight of the first component system be present. Generally it is preferred that no water be present in the first component so that the first component is anhydrous. However, small amounts of water from about 1 to 5% by weight of the system can be tolerated.

The second component system of this invention provides the liquid aqueous carrier medium for the cosmetic composition. The liquid aqueous carrier medium which constitutes the second component can be water or it can be an oil in water or oil in water liquid emulsion. Providing a second component system separate from the first and third component systems until the final formulation of the cosmetic composition prevents the water present in the second component from adversely affecting the ingredients in the first and second components. In accordance with this invention, any cosmetically acceptable carrier which is compatible with the water can be combined with water to form the aqueous carrier medium of the second component system. The aqueous carrier medium can contain one or more oil components. The oil component may be any pharmaceutically or cosmetically acceptable oily material which is substantially insoluble in water. These materials can be found for example in the CTFA International Dictionary of Cosmetic Ingredients as well as the U.S. Pharmacopoeia or other equivalent sources. Suitable oil components include, but are not limited to, natural oils, such as coconut oil; hydrocarbons, such as mineral oil and hydrogenated polyisobutene; fatty alcohols, such as octyldodecanol; esters, such as C12-15 alkyl benzoate; diesters, such as propylene glycol dipelargonate; triesters, such as glyceryl trioctanoate; sterol derivatives, such as lanolin; animal waxes, such as beeswax, plant waxes, such as carnauba; mineral waxes, such as ozokerite; petroleum waxes, such as paraffin wax; synthetic waxes, such as polyethylene; and mixtures thereof.

The second component system may contain in addition to the oils stable ingredients which include various antioxidants, stabilizers, moisturizers, preservatives and emulsifiers such as those mentioned hereinbefore. Any conventional antioxidant, moisturizer, stabilizer, preservative, or emulsifier which is stable in the liquid aqueous carrier medium can be present in the second component. In general, the second component system containing the liquid aqueous carrier medium for the cosmetic composition should be present in the kit in an amount of from about 10% to about 40% by weight of the formulated the cosmetic composition produced there from.

To the first and second component systems there may be added known conventional cosmetic additives, adjuvants and vehicle substrates. Emulsifiers such as glycerol stearate or methyl glucose sesquistearate may also be present Also present may be organic solvents, such as lower aliphatic alcohols from 1 to 6 carbon atoms, such as ethanol isopropanol or propanol, or glycols such as glycerin or 1,2-propylene glycol. The following can also be included based upon the weight of the final cosmetic product: perfume oils in a quantity of from about 0.1%, to 5%, by weight of opacifiers such as ethylene glycol distearate in a quantity of from about 0.2%, to about 5%, by weight of a wetting agents or emulsifiers selected from the classes of anionic, cationic, amphoteric or nonionic surfactants, such as fatty alcohol sulfates, alkyl benzene sulfonates, alkyltrimethylammonium salt; alkyl betaines, ethoxylated fatty alcohols, ethoxylated nonyl phenols, ethoxylated fatty acid esters in a quantity of from about 0.1%, to about 5%, by weight as well as anti-bacterial and fungicidal ingredients; thickeners (such as bentonite); pH buffer substances; moisture retaining agents; fragrances or perfumes; perfume oils; colorants (such as natural or synthetic direct dyes but also tinting agents such as fluorescein sodium salt); sunscreens or UV filters; preservatives; antioxidants (such as tocopherols); pyrogemic silicic acid; complexing agents; and also physiologically tolerable inorganic or organic acids, such as phosphoric acid, acetic acid, formic acid, glyoxylic acid, lactic acid, tartaric acid or citric acid; bases; salts (such as sodium chloride); buffers (such as sodium citrate or sodium phosphate); consistency-lending agents; and natural, modified, partly or entirely synthetic polymers (such as chitosan, FMOC chitosan and PVP). Naturally, one skilled in the art will be familiar with which of the various individual adjuvants and vehicle substances to be added in order to obtain a desired formulation of the topical cosmetic preparation of this invention.

The third component of the kit which is used for making the cosmetic composition is a thickening or gelling agent or combination of such agents. This agent can be any of the conventional thickening or gelling agents utilized in forming cosmetic creams or lotions. Suitable hydrophilic gelling or thickening agents can generally be described as water soluble or colloidally water soluble polymers and include cellulose, ethers, hydroxyethyl cellulose, hydroxypropylmethyl cellulose. Among suitable hydrophilic gelling or thickening agents are acrylic acid/alkyl acrylate copolymers and the carboxyvinyl polymers sold by the B.F. Goodrich Company under the trade mark of Carbopol resins. These resins consist essentially of a colloidally water-soluble polyalkenyl polyether cross linked polymer of acrylic acid cross linked with from 0.75% to 2.00% by weight of the polymer of a cross linking agent such as for example polyallyl sucrose or polyallyl pentaerythritol. Examples include Carbopol 934, Carbopol 940, Carbopol 950, Carbopol 980, Carbopol 951 and Carbopol 981. Carbopol 934 is a water-soluble polymer of acrylic acid cross linked with about 1% by weight of the polymer of a polyallyl ether of sucrose having an average of about 5.8 allyl groups for each sucrose molecule. Also suitable for use herein are hydrophobically-modified cross-linked polymers of acrylic acid having amphipathic properties available under the Trade Name Carbopol 1382, Carbopol 1342 and Pemulen TR-1 (CTFA Designation:Acrylates/10-30 Alkyl Acrylate Crosspolymer). A combination of the polyalkenyl polyether cross-linked acrylic acid polymer and the hydrophobically modified cross-linked acrylic acid polymer is also suitable for use herein.

Among the preferred gelling or thickening agents for use in this invention are the anionic acrylic polymers and cross-linked acrylic copolymers that are generally utilized in cosmetic compositions. These acrylates are specifically disclosed in the International Journal of Toxicology, 21 (Suppl. 3): 1-50, 2002, Cosmetic Ingredient Review. Among the preferred anionic acrylate polymers and acrylate cross-linked copolymers for use as thickening agents are the following:

1. Hydroxyethyl Acrylate/Sodium Acrilodimethyl Taurate Copolymer/Squalene/Polysorbate 60 (Simulgel NS—Seppic Inc.)

2. Polyacrylamine/C₁₃₋₁₄ Isoparafin Laureth-7 (Sepigel 305—Seppic Inc.)

3. Ammonium Polyacrylate/Isohexdecane/PEG-40 Castor Oil (Simulgel A—Seppic Inc.)

4. Sodium Acrylate/Sodium Acrylodimethyl Taurate Copolymer/Isohexadecane/Polysorbate 80 (Simulgel EG—Seppic Inc.)

5. C₁₃₋₁₄ Isoparaffin/Mineral Oil/Sodium Polyacrylate/Polyacrylamide/Polysorbate 80 (Sepigel 501—Seppic Inc.)

6. Steareth-10 Allyl Ether/Acrylates Copolymer (Salcare SC80—Ciba Inc.)

7. Acrylates Copolymer (Salcare SC81—Ciba Inc.; Rheocare TTN and Rheocare TTN₂—Seaview Technologies Inc.)

The three component systems containing the various ingredients are placed in the kit in two or three separate compartments. In accordance with a preferred embodiment they are packaged separately in the kit so that none of the contents of these component systems come in contact with each other. When it is desired to produce the cosmetic composition the three component systems are mixed together by placing the component systems in the mixing vessel supplied with the kit. In the kit the third component is generally in an amount of from about 1.0% to 15% by weight of the total cosmetic composition formed. If it is desired to produce a lotion, generally the third component is provided only in an amount of from 1.0% to 5.0% by weight of the total composition. On the other hand, if one wishes to prepare a cream usually the third component is provided in an amount higher than 5% by weight of the final cosmetic composition. Generally, the three packets are provided in the kit to produce upon mixture a topical liquid cosmetic product of from about 5 to 400 g when these component systems are mixed in the mixing vessel. In the kit, the component systems as outlined above can be provided in two or three separate packages. The packages may be interconnected or joined together by separable perforations such as perforations that can be torn by the hand or cut with a scissor. In this manner, the individual packets containing these component systems can be separated, opened and poured directly into the mixing vessel to produce the desired topical liquid cosmetic product in a simple manner. Each of the component systems can then be placed in the mixing vessel and stirred lightly to produce a ready to use topical liquid cosmetic product in the form of a cream. lotion, oil or other liquid topical cosmetic product which can be applied directly from the mixing vessel to the body such as face, nails, hair, neck or throat. The cosmetic compositions that can be prepared include face creams, hand and throat creams, nail cream, day or night cream, shampoos, conditioners, etc.

In addition, in accordance with another embodiment of this invention, the kit can contain additional component systems or packages In this case there can be multiple sets of two or three packages described hereinbefore ,each for preparing different cosmetic preparations and each set containing the cosmetically active component system, the aqueous cosmetic base medium and the thickening component. Among the most common sets of cosmetic preparation are one for preparing a day cream, and the other for preparing a night cream. The night cream and day cream can be each in two or three separate packets. On the other hand, where the thickening agent is in a separate container from the other two component systems, the thickening agent included with the night or day packets can be used for preparing both creams. In this case, a portion can be utilized to thicken the night cream and another portion utilized to thicken the day cream depending upon the desired thickening of both. If a large quantity is made, the creams can be stored in the mixing jar in the refrigerator for a few days without substantial loss of activity.

In accordance with the preferred embodiment of this invention, a liquid component system containing liquid forms or liquid extracts of natural food and beverage materials or mixtures thereof can be added to these cosmetic compositions by blending this component system into the mixture of the other three components. In this manner, the cosmetic can contain natural components or mixtures thereof which are obtained from food and beverages. By adding this component prior to applying the cosmetic, one produces a cosmetic having these skin effective natural ingredients which work in synergy with the active ingredients without the danger of loss of activity through such things as deterioration. Deterioration is common problem with such natural materials. As one knows, these natural ingredients while great for the skin are also sensitive to air and heat. Providing a cosmetic composition with these natural materials which is prepared just before being applied to the skin, allows these natural ingredients to be incorporated into the cosmetic composition without the danger of substantial decomposition. In addition, this allows one to tailor make the precise natural ingredients into a cosmetic composition that one wishes or needs to apply to the skin. Furthermore, these natural ingredients work in synergy with the active ingredients present in the other components of this product

Any conventional liquid form or extract of a food or beverage such as a juice or other type of liquid forms or extracts from a food or beverage can be utilized. These include liquid extracts from fruits, vegetables, or herbs as well as liquids obtained from milk products, wines, tea, coffee, beer etc, all of which can be incorporated into the cosmetic composition for application to the human body. The tea extracts that are commonly utilized are the chamomile, green tea, etc. which are a source of phenol antioxidants. Dairy products such as buttermilk and yogurt are good sources of alpha hydroxy acids. Liquid vegetable extracts are also useful and helpful. Water can be added to the composition, as well as juices of fruits such as oranges, grapefruits, lemons, pineapples, apples, peaches, strawberries, etc. since they are a good source of vitamins, phytochemicals, flavonoids, phenols, indols, carotenoids. Vegetable extracts include juices from broccoli, carrots, onion, peppers, spinach and tomatoes. All of these can be utilized in mixtures to provide a desired skin treatment. In general, these liquid natural component systems are added to the mixing vessel upon mixing the three component systems obtained from the packages within the kit. These component systems can be tailored by the individual in any desired manner. In general, the fourth component is blended into the components in the mixing vessel in an amount sufficient to provide from 20% to 60% by weight of the cosmetic composition produced through said mixing.

In accordance with this invention the liquid topical cosmetic product of this invention may be obtained by pouring the thickening agent and the cosmetically active ingredients into the mixing vessel. Half the active base present as the third component may be added and the mixture stirred lightly for about 10 seconds or longer. After this period, the second half of the active base is added and stirred. After stirring for a few minutes the fourth component containing the water and natural extracts can be added to the mixture. Stirring or blending these natural materials produces a the final liquid cosmetic product that is ready for topical application to the human body.

Among the preferred day creams utilized in moisturizing treatment are creams prepared from ingredients contained within the following four component systems set forth in Table I. The first three component systems are different and separately packaged. The ingredients set forth in the Tables are the preferred ingredients for use in these component systems. In accordance with a preferred embodiment, the component systems may contain one or blends of these ingredients depending upon the nature of the cosmetic desired. After mixing the three component systems, the mixture is blended with the natural materials of the fourth component system. In Table I the levels are % by weight, based upon the weight of component system in which the ingredient is found. These systems when formulated produce various desired cosmetic compositions.

TABLE I
MOISTURIZING DAY CREAM

| | |
|---|---|
| Function | Levels |

COMPONENT SYSTEM₃ - THE THICKENER

Ingredients

| | | |
|---|---|---|
| Anionic Acrylic Polymer | Thickener, emulsifier | 0.1-10% |
| Cross-Linked Acrylic | Thickener, emulsifier | 0.1-10% |
| Copolymer | | |
| Fatty Acid Amides | Thickener | 0.1-10% |

COMPONENT SYSTEM₁ -
THE COSMETICALLY ACTIVE MEDIUM

| | | |
|---|---|---|
| Corn Oil | Diluent | 20-70% |
| Sunflower Oil | Natural Moisturizer | 10-20% |
| Vitamin E (Mixed | Natural Protectant, | 0.5-15% |
| Tocopherols) | Antioxidant | |
| Jojoba Oil | Natural Moisturizer | 3-10% |
| Lycopene | Natural Antioxidant | 0.01-0.25% |
| Lutein | Natural Antioxidant | 0.01-0.25% |
| Tocotrienol | Natural Antioxidant | 0.05-0.15% |

COMPONENT₂ -
THE AQUEOUS COSMETIC ACTIVE BASE MEDIUM

| | | |
|---|---|---|
| Mineral Oil | Diluent, Moisturizer | 1-10% |
| Diisopropyl Adipate | Moisturizer | 0.2-3% |
| Glyceryl Stearate | Emulsifier | 1-5% |
| Methyl Glucose | Emulsifier | 0.5-5% |
| Sesquistearate | | |
| Shea Butter | Natural Moisturizer | 0.1-10% |
| Squalane | Humectant, Moisturizer | 0.1-10% |
| Cetearyl Octanoate | Moisturizer | 0.1-1% |
| Vitamin E Acetate | Natural, In Vivo | 0.1-15% |
| | Antioxidant | |
| Vitamin C or Derivative | Antioxidant | 0.1-15% |
| Glycerin | Moisturizer, Humectant | 0.1-10% |
| Panthenol | Moisturizer, Humectant | 0.1-10% |
| Carbomer | Stabilizer, Thickener | 0.1-0.5% |
| Disodium EDTA | Chelating Ingredient | 0.1-0.5% |
| Quaternium-15 | Preservative | 0.1-0.5% |
| Methylparaben | Preservative | 0.1-1.0% |
| Propylparaben | Preservative | 0.1-1.0% |
| Butylparaben | Preservative | 0.1-1.0% |
| Water | Solvent | 10-80% |

COMPONENT SYSTEM₄ FRESH NATURAL ADDITIVES

| | | |
|---|---|---|
| Fresh Natural Additives | | |
| Pure Mineral Water | Diluent, Solvent | 20-60% |
| Green Tea | Diluent, Rich in Antioxidants | 20-60% |
| Chamomile Tea | Diluent, Soothing | 20-60% |
| Apple Juice | Diluent, Rich in Isoflavonoids | 20-60% |
| | and other Antioxidants | |
| Watermelon | Diluent, Rich in Vitamin C, | 20-60% |
| | Lutein, Beta Carotene, Lycopene | |
| | (Natural Antioxidants) | |
| Tomato Juice | Diluent, Rich in Vitamin C, | 20-60% |
| | Lycopene, Lutein (Natural | |
| | Antioxidants) | |
| Cherries, Berries & Beets | Diluent, Rich in vitamins, | 20-60% |
| | minerals, antioxidants, Salicylic | |
| | Acid, Flavonoids, | |
| | Phytoestrogens, Anthocyanins | |
| Red Wine | Diluent, Rich in Polyphenols | 20-60% |
| | (Antioxidants) | |
| Grape Juice | Diluent, Rich in Polyphenols | 20-60% |
| | (Antioxidants) | |

Among the preferred night creams utilized in corrective treatment are creams prepared from ingredients contained within the following four component systems set forth in Table II. The first three systems are separately packaged and the component systems in each are different. The ingredients set forth in the Table II are the preferred ingredients for use in these component systems. In accordance with a preferred embodiment, the component systems may contain one or blends of these ingredients depending upon the nature of the cosmetic desired. After mixing the three component systems, the mixture is blended with the natural materials of the fourth component system. In Table II, the levels are % by weight, based upon the weight of component system in which the ingredient is found. These when formulated produce various desired cosmetic compositions

TABLE II
NIGHT CREAM

| | |
|---|---|
| Function | Levels |

COMPONENT SYSTEM₃ THICKENER

Ingredients

| | | |
|---|---|---|
| Anionic Acrylic Polymer | Thickener, Emulsifier | 0.1-10% |
| Cross-Linked | Thickener, Emulsifier | 0.1-10% |
| Acrylate Copolymer | | |

COMPONENT SYSTEM₁ -
COSMETICALLY ACTIVE MATERIALS

| | | |
|---|---|---|
| Corn Oil | Diluent, Humectant | 20-70% |
| Jojoba Oil | Natural Moisturizer, Humectant | 10-20% |
| Vitamin A Derivatives | Anti-Wrinkling | 1-10% |
| Sesame Oil | Natural Moisturizing Oil, | 0.1-7% |
| | Humectant | |
| Sweet Almond Oil | Non-greasy Moisturizing Oil, | 1-7% |
| | Humectant | |
| Apricot Kernal Oil | Non-greasy Moisturizing Oil, | 1-7% |
| | Humectant | |
| Vitamin E Alcohol | Antioxidant | 1-5% |
| Tocotrienol | Natural Antioxidant | 1-15% |

COMPONENT SYSTEM₂ -
AQUEOUS ACTIVE COSMETIC BASE

| | | |
|---|---|---|
| Mineral Oil | Diluent, Moisturizer | 1-10% |
| Steareth-2 | Emulsifier | 1-5% |
| Cetyl Alcohol | Thickener, Moisturizer | 0.5-3% |
| Diisopropyl Adipate | Moisturizer | 0.2-3% |
| PPG-15 Stearyl Ether | Emulsifier | 0.2-3% |
| Vitamin E Acetate | In Vivo Antioxidant, | 0.1-15% |
| | Moisturizer | |
| Polyoxyethylene-20 | Emulsifier | 0.2-3% |
| Cetearyl Octanoate | Moisturizer | 0.1-2% |
| Dimethicone | Smoothing Ingredient | 0.1-1% |
| Niacinamide | Vitamin B₃ | 0.2-3% |
| Panthenol | Moisturizer, Humectant | 0.1-10% |
| Carbomer 980 | Stabilizer, Thickener | 0.1-0.5% |
| Disodium EDTA | Chelating Material | 0.1-0.5% |
| Aminomethyl Propanol | PH Adjuster | 0.1-0.5% |
| Quaternium-15 | Preservative | 0.1-0.5% |
| Methylparaben | Preservative | 0.1-1.0% |
| Propylparaben | Preservative | 0.1-1.0% |
| Butylparaben | Preservative | 0.1-1.0% |
| Water | Solvent | 10-80% |

COMPONENT SYSTEM 4

| | | |
|---|---|---|
| Fresh Natural Additives | Function | Levels |
| Pure Mineral Water | Diluent, Solvent | 20-60% |
| Green Tea | Diluent, Rich in | 20-60% |
| | Antioxidants | |
| Chamomile Tea | Diluent, Soothing | 20-60% |
| Soy Milk | Diluent, Rich in | 20-60% |
| | Phytoestrogens, Flavonoids | |
| Apple Juice | Diluent, Rich in Flavonoids, | 20-60% |
| | Antioxidants | |
| Grapefruit Juice | Diluent, Rich in Vitamin C, | 20-60% |
| | Isoflavones | |
| Coffee | Diluent, Rich in Caffeine | 20-60% |
| Cranberry Juice | Diluent, Rich in Vitamin C | 20-60% |

### EXAMPLE 1

Product Name: Moisturizing Day Cream

A moisturizing three component treatment system day cream was formulated from three separate packages each containing a different system of components as follows.

Part I: Actives

In forming the Actives component for use in the finished product a mixture of the following ingredients was prepared. From this mixture 2 grams were taken and used as the Actives composition in 30 grams of a cream.

| Ingredients | INCI Designation | % W/W |
|---|---|---|
| Corn Oil | Corn Oil | 54.94 |
| Sunflower Oil | Sunflower Oil | 15.00 |
| Gamma Tocopherol | Tocopherol | 10.00 |
| Jojoba Oil | Jojoba Oil | 5.00 |
| Vitamin C | Ascorbic Acid | 15.00 |
| Lycopene 10% FS** | Lycopene (and) | 0.03 |
| | Corn Oil | |
| Lutein 20% FS*** | Lutein (and) Corn Oil | 0.03 |

**Roche Vitamins Inc.
***Roche Vitamins Inc.

Procedure:

Blend until uniform (disperse Vitamin C).

Part II: Base

In forming the base component for use in the finished product a mixture of the following products was prepared. From this mixture 10 grams were taken and used as the base in the finished product of 30 grams.

| | | % |
|---|---|---|
| Ingredients | INCI Designation | W/W |
| Part 1 | | |
| Klearol Mineral Oil (Witco) | Mineral Oil | 6.00 |
| Ceraphyl 230 (ISP) | Diisopropyl Adipate | 3.00 |
| D-alpha Tocopheryl Acetate | Tocopheryl Acetate | 3.00 |
| Emerest 2400 (Cognis) | Glyceryl Stearate | 3.00 |
| Glucate SS (Amerchol) | Methyl Glucose Sesquistearate | 3.00 |
| Glucamate SSE-20 (Amerchol) | Methyl Glucate-20 | 2.50 |
| Shea Butter | Shea Butter | 3.00 |
| Crodamol CAP (Croda) | Cetearyl Octanoate | 1.00 |
| Fitoderm (Centerchem) | Squalane | 1.00 |
| Propylparaben | Propylparaben | 0.10 |
| Part 2 | | |
| Deionized Water | Water | 60.20 |
| Glycerine | Glycerin | 6.00 |
| D-Panthenol | Panthenol | 3.00 |
| Carbopol 980 (Noveon) | Carbomer | 0.30 |
| Versene NA2 (Dow Chemical) | Disodium EDTA | 0.60 |
| Methylparaben | Methylparaben | 0.10 |
| Part 3 | | |
| Sodium Hydroxide, 10% | Sodium Hydroxide | 1.20 |
| Part 4 | | |
| Dowicil 200 (Dow Chemical) | Quaternium 15 | 0.90 |
| Deionized Water | Water | 3.00 |

Procedure:

Step 1—Mix ingredients of Part 1 and heat to 70° C.

Step 2—Dissolve the Panthenol and Glycerin in Water. Sprinkle the Carbomer 980 and mix until dispersed. Add Disodium EDTA and mix. Heat to 75° C.

Step 3—Add Part 2 into Part 1 with moderate mixing.

Step 4—Add Part 3 with mixing. Cool to 45° C.

Step 5—Add Part 4. Cool to RT with slow agitation.

Part III: Thickener

2.25 grams of the Thickener were used in the finished product.

| Ingredient | INCI Designation |
|---|---|
| Simulgel NS* | Hydroxyethylacrylate/Sodium acryloyl |
| | Dimethyltaurate copolymer & Squalane |
| | & Polysorbate 60 |

*Seppic, Inc.

Part IV: Suggested Fresh Natural Additive

Fresh Natural Additives

15.75 grams of brewed Green Tea were added to Parts I, II and III and mixed until smooth and uniform, to form 30 grams. of a cream.

Finished Product/Per 30 grams

| Components | % W/W |
|---|---|
| Activator | 6.66 |
| Active Drops | 6.66 |
| Active Base | 33.33 |
| Fresh Natural Additives | 53.35 |

### EXAMPLE 2

Product Name: Corrective Night Cream

Part I: Actives

In forming the Actives component for use in the finished product a mixture of the following ingredients was prepared. From this mixture 2 grams were taken and used as the Actives composition in 30 grams of a cream.

| | | % |
|---|---|---|
| Ingredients | INCI Designation | W/W |
| Corn Oil | Corn Oil | 53.18 |
| Jojoba Oil | Jojoba Oil | 15.00 |
| Vitamin A Palmitate P1.7 | Retinyl Palmitate | 3.82 |
| (Roche Vitamins) | | |
| Vitamin A Acetate | Retinyl Acetate | 3.00 |
| Vitamin A Alcohol | Retinol | 2.00 |
| Sesame Oil | Sesame Oil | 5.00 |
| Sweet Almond Oil | Sweet Almond Oil | 5.00 |
| Apricot Kernel Oil | Apricot Kernel Oil | 5.00 |
| Vitamin E Alcohol | Tocopherol | 4.00 |
| Vitamin C | Ascorbic Acid | 4.00 |

Procedure:

Blend until uniform (disperse Vitamin C).

Part II: Base

In forming the base component for use in the finished product a mixture of the following products was prepared. From this mixture 10 grams were taken and used as the base in the finished product of 30 grams.

| | | % |
|---|---|---|
| Ingredients | INCI Designation | W/W |
| Part 1 | | |
| Drakeol 9 Light Mineral Oil | Mineral Oil | 6.00 |
| (Penreco) | | |
| Brij 72 (Noveon) | Steareth-2 | 2.50 |
| Lanette 16 (Cognis) | Cetyl Alcohol | 2.00 |
| Ceraphyl 230 (ISP) | Diisopropyl Adipate | 3.00 |
| Arlamol E (Uniqema) | PPG-15 Stearyl Ether | 3.00 |
| D-alpha Tocopheryl Acetate | Tocopheryl Acetate | 3.00 |
| Arlasolve 200L (Uniqema) | Polyoxyethylene 20 Isohexadecyl | 3.00 |
| | Ether (and) Water | |
| Propylparaben | Propylparaben | 0.10 |
| Crodamol CAP (Croda) | Cetearyl Octanoate | 1.00 |
| Dow Corning 200 Fluid, 350 | Dimethicone | 1.50 |
| cs | | |
| Part 2 | | |
| Deionized Water | Water | 66.60 |
| D-Panthenol | Panthenol | 3.00 |
| Carbopol 980 (Noveon) | Carbomer 980 | 0.30 |
| Versene NA2 (Dow | Disodium EDTA | 0.60 |
| Chemical) | | |
| Methylparaben | Methylparaben | 0.10 |
| Part 3 | | |
| AMP (Angus) | Aminomethyl Propanol | 0.30 |
| Deionized Water | Water | 0.10 |
| Part 4 | | |
| Dowicil 200 (Dow Chemical) | Quaternium 15 | 0.90 |
| Deionized Water | Water | 3.00 |

Procedure:

Step 1—Mix ingredients of Part 1 and heat to 70° C.

Step 2—Dissolve the Panthenol in Water. Sprinkle Carbomer 980. Add Disodium EDTA and mix. Heat to 75° C.

Step 3—Add Part 2 into Part 1 with moderate mixing.

Step 4—Add Part 3 with mixing. Cool to 45° C.

Step 5—Add Part 4. Cool to RT with slow agitation.

Part III: Thickener

2.25 grams of the Thickener were used in the finished product.

| Ingredient | INCI Designation | % W/W |
|---|---|---|
| Simulgel NS* | Hydroxyethyl Acrylate/Sodium | 100.00 |
| | Acryloyl | |
| | Dimethyltaurate Copolymer & | |
| | Squalane & Polysorbate 60 | |

*Seppic, Inc.

Part IV: Suggested Fresh Natural Additive

15.75 grams of unflavored Soy Milk were added to Parts I, II and III and mixed until smooth and uniform, to form 30 grams of a cream.

Finished Product/Per 30 grams

| Components | % W/W |
|---|---|
| Activator | 6.66 |
| Active Drops | 6.66 |
| Active Base | 33.33 |
| Fresh Natural Additives | 53.35 |

### EXAMPLE 3

Product Name: Scalp Conditioning Shampoo

Part I: Actives

In forming the Actives component for use in the finished product a mixture of the following ingredients was made. From this mixture 2 grams were taken and used as the Actives composition in 240 grams of the Shampoo.

| | INCI Designation | % W/W |
|---|---|---|
| Ingredients | | |
| Vitamin A Palmitate P1.7 | Retinyl Palmitate (and) Corn Oil | 35.30 |
| (Roche Vitamins) | Tocopheryl Acetate | 3.00 |
| Vitamin E Acetate | | |
| Corn Oil | Corn Oil (Zea Mays) | 62.00 |
| Procedure: | | |
| Blend until uniform. | | |

Part II: Base

In forming the base component for use in the finished product a mixture of the following ingredients was prepared. From this mixture 28 grams were taken and used as the base composition in 240 grams of finished product.

| Ingredients | INCI Designation | % W/W |
|---|---|---|
| Part 1 | | |
| TEA-Lauryl Sulfate | TEA-Lauryl Sulfate | 10.8 |
| Sodium Myreth Sulfate | Sodium Myreth Sulfate | 11.3 |
| Cocamide DEA | Cocamide DEA | 6.2 |
| Lauramide DEA | Laurarmide DEA | 7.3 |
| Phytantriol | Phytantriol | 0.8 |
| Methylparaben | Methylparaben | 2.0 |
| Propylparaben | Propylparaben | 1.0 |
| Part 2 | | |
| Deionized Water | Water | 15.0 |
| Part 3 | | |
| D-Panthenol | Panthenol | 2.4 |
| Ethyl Panthenol | Panthenyl Ethyl Ether | 1.2 |
| Deionized Water | Water | 35.0 |
| Part 4 | | |
| Citric Acid | Citric Acid | 0.6 |
| Deionized Water | Water | qs to 100.0 |

Procedure:

Step 1—Weigh items of Part 1 into a steam jacketed kettle and heat to 55° C. until uniform and clear. Cool to 45° C.

Step 2—Add Deionized Water and stir gently.

Step 3—Add Part 3 items and stir.

Step 4—Add items of Part 4.

Step 5—Q.s. with Deionized Water

Part III: Thickener

5 grams of the Thickener were used in the finished product.

| Ingredients | % W/W |
|---|---|
| Sodium Chloride | 5.0 |
| Water | 95.0 |

Procedure:

Mix to dissolution.

Part IV: Suggested Fresh Natural Additive

205 grams of pure mineral water were added to Pats I, II and III and mixed to form 240 grams of a shampoo.

What is claimed:

1. A method of applying a liquid topical cosmetic composition to the human body comprising:
1) providing three component systems which when mixed together form a cosmetic cream or lotion for application to human skin, said component system being:
a) a first component system containing a mixture of cosmetically active ingredients,
b) a second component system containing a liquid aqueous cosmetic carrier medium, and
c) a third component system containing a thickening agent which when added to said liquid medium forms a lotion or cream;
with at least said second component system being separated from said first component system;
2) mixing said component systems in a mixing vessel in a predetermined ratio to form said liquid cosmetic product; and
3) topically applying from said mixing vessel to said body said cosmetic product formed in said mixing vessel.

2. The method of claim 1, wherein said cosmetically active component system contains an anti-wrinkling agent, moisturizer, vitamin, antioxidant, skin nutrient or mixtures thereof.

3. The method of claim 1, wherein the component systems are mixed in predetermined amounts to form from about 5 grams to 400 grams of the cosmetic in said mixing vessel.

4. The method of claim 3, wherein said component systems are provided in the following percentages by weight based upon the total weight of said cosmetic composition
1) first component system provided in the amount of from 0.5 to 20%;
2) second component system provided in the amount of from 10% to 40%; and
3) third component system provided in the amount of from 1% to 15%.

5. The method of claim 1, wherein the first and third component systems are provided in the mixing vessel in a single package.

6. The method of claim 1, wherein each of said component systems are provided in three separate packages.

7. The method of claim 6, wherein said cosmetically active component system contains an anti-wrinkling agent, moisturizer, vitamin, antioxidant, skin nutrient or mixtures thereof.

8. The method of claim 6, wherein the component systems are mixed in predetermined amounts to form from about 5 grams to 400 grams of the cosmetic in said mixing vessel.

9. A method of applying a liquid topical cosmetic composition topically to the human body comprising:
1) providing three component systems which when mixed together form a cosmetic cream or lotion for application to human body, said component system being:
a) a first component system containing a mixture of cosmetically active ingredients,
b) a second component system containing a liquid aqueous cosmetic carrier medium, and
c) a third component system containing a thickening agent which when added to said liquid medium forms a lotion or cream;
with at least said second component system being separated from said first component system;
2) mixing said component systems in a mixing vessel in a predetermined ratio to form said liquid cosmetic product;
3) adding to the mixture of said three components in said mixing vessel, a fourth liquid component containing liquid forms or liquid extracts of food and beverage materials or mixtures thereof; and
4) applying from said mixing vessel topically to said body said liquid cosmetic product containing said fourth component.

10. The method of claim 9, wherein the first and third component systems are provided in the mixing vessel in a single package.

11. The method of claim 10, wherein each of said component systems are provided in three separate packages.

12. The method of claim 11, wherein said liquid form and extracts are obtained from fruit juices, coffee extracts, wine, tea extracts, vegetable juices or mixtures thereof.

13. The method of claim 11, wherein said active ingredient is an anti-wrinkling agent, moisturizer, vitamin, antioxidant, skin nutrient or mixtures thereof.

14. The method of claim 13, wherein the component systems are mixed in predetermined amounts to form from about 5 grams to 400 grams of the cosmetic in said mixing bowl.

15. The method of claim 14, wherein said component systems are provided in the following percentages by weight based upon the total weight of said cosmetic composition
1) first component system provided in the amount of from 0.5 to 20%;
2) second component system provided in the amount of from 10% to 40%;
3) third component system provided in the amount of from 1% to 15%; and
4) fourth component system provided in the amount of from 20% to 60%.

16. A kit for forming a topical liquid cosmetic composition comprising two or three separated containers each containing a predetermined amount of different component systems, such that all of said component systems when mixed together form said cosmetic composition for direct topical application, with one of said component systems containing a mixture of active cosmetic ingredients and another of said component system separated from the other component systems in the kit containing a liquid aqueous cosmetic carrier medium, and a mixing vessel for mixing said component systems in a predetermined ratio to form said composition.

17. The kit of claim 13, being two separate containers, one of said containers containing an aqueous carrier medium for the liquid cosmetic composition and the other container containing a mixture of cosmetically active ingredients and a thickening agent for said aqueous carrier medium.

18. The kit of claim 16 comprising three separate containers wherein:
a) the component system in the first container contains a mixture of cosmetically active ingredients,
b) the component system in the second container contains a liquid aqueous cosmetic carrier medium, and
c) the component system of the third container contains a thickening agent which when added to said other two component systems forms said topical liquid cosmetic composition.

19. The kit of claim 19, wherein the component systems are present in the containers each in a predetermined amount so as to form from about 5 grams to about 400 grams of the cosmetic composition when mixed in said mixing vessel.

20. The kit of claim 19, wherein said component systems are provided in each of the containers in the following percentages by weight based upon the weight of the total cosmetic composition produced in said mixing vessel:
1) the component system in the first container being in an amount of from about to about 0.5 to 20%;
2) the component system in the second container being in an amount of from about 10% to about 40%; and
3) the component system in the third container being in an amount of from about 1% to about 15%.

21. The kit of claim 20 wherein each of the three separated containers is a separated package.

22. The kit of claim 21 wherein each of the three separated packages are joined together via separable perforations.
